(19) United States
(12) Patent Application Publication
(10) Pub. No.: US 2019/0269198 A1
(43) Pub. Date: Sep. 5, 2019

(54) SUPPORT STRUCTURE OF A FOOT ARTICULATION AND SHOE WHICH COMPRISES SAID SUPPORT STRUCTURE

(71) Applicants: Armanda BOCCARDO, (US); Alessandro LAZZERONI, (US)

(72) Inventor: Armanda BOCCARDO, San Giuliano Terme (PI) (IT)

(73) Assignees: Armanda BOCCARDO, San Giuliano Terme (PI) (IT); Alessandro LAZZERONI, Pontedera (PI) (IT)

(21) Appl. No.: 15/571,115

(22) PCT Filed: May 2, 2016
(86) PCT No.: PCT/IB2015/000564

§ 371 Date: Nov. 1, 2017

(30) Foreign Application Priority Data
May 4, 2015 (IT) PI2015A000035

(51) Int. Cl. A43B 7/24; A61F 5/01; A43B 13/18; A43B 13/20; A61F 5/34

(52) U.S. Cl. CPC A43B 7/24; A61F 5/012; A61F 5/34; A43B 13/20; A61F 5/0111; A43B 13/189

(57) ABSTRACT

Support structure (1) of a foot articulation that comprises a body in which a foot articulation can be arranged, a lung body (3), associated with the body, which defines a deformable chamber (4). The lung body comprises a first portion (5) which extends in a direction substantially longitudinal and a second portion (6) which extends in a direction substantially orthogonal with respect to the first portion arranged between the body and the ankle of the foot articulation, such that when occurs a foot support inclined at an angle with respect to a ground plane, is generates a first force on said first portion defined by a first vector (T1) substantially orthogonal with respect to said ground plane (P) that causes—in feedback—a subsequent expansion of said second portion which generates a second force, defined by a second vector (T2) substantially parallel to said ground plane, that is adapted to provide a support for ankle.

### FIELD OF THE INVENTION

The present invention relates to a support structure of a foot articulation that is adapted to prevent foot or ankle injuries during sports activities or physic activities in which deambulation movements are done.

Furthermore, the invention relates to a shoe that comprises said support structure.

### STATE OF THE ART

As well known, exist foot brace or belts that is adapted to support and locally protect a foot articulation. These are conformed in the form of stiff elements made in plastic material or belts, resilient and not, which are arranged on a portion of the foot and/or of the ankle, in order to provide an independent protection from the shoes.

In particular, a foot brace allows to provide a stiffening of the foot articulation in order to limit movements of it, and to keep in a steady position the foot-ankle articulation. These are mainly used in a post trauma phase, in order to allow a foot articulation rehabilitation.

The foot brace has a stiff structure adapted to keep fastened the foot articulation and this structure is not adapted to a normal deambulation, since are not much comfortable.

To overcome this drawback are known, also, foot brace that comprise damper portions defined by at least one closed chambers—filled with a fluid such as air or gel—which carry out a damper function. This way, the foot brace in addition to the support function ensures also a damper function in order always to protect the foot articulation by the actions, which is subject during the deambulation, and in order to increase the comfort.

In addition to the foot brace, shoes that increase a protection side of the foot articulation are known, which is adapted to obtain a static support of it, in order to limit the transversal movement, which causes foot sprain.

However, the above described products according to the prior art provide a static protection with a permanent stiff conformation which is present in an independent way from the presence of actions potentially dangerous for foot and/or the ankle and, and then these products are not adapted to sports activities since are not much comfortable and with a stiff conformation.

In WO2013044894 a device for preventing or avoiding the thrombosis, in particular in lower limbs veins is described. U.S. Pat. No. 4,502,470 describes a device conformed to be actuated for the venous insufficiency.

Structurally the two devices comprise a base pillow, which is arranged under a foot sole, which is fluidically connected to a side pillow that extends in height starting from said base pillow. The base pillow and the side pillow defining respective chambers, in a fluid connection to each other, and which are filled with a gas.

The object is to produce, during the gait of the patient, which puts the weight on the foot sole, a normal flow of blood, which adjusts the cardio circulatory apparatus.

The devices of which above, moreover is able to act on the ankle apparatus articulation, in order to avoid distortions of the foot sole, during sport activities, when it doesn't adhere steadily to the ground.

### SUMMARY OF THE INVENTION

It is then a feature of the present invention to provide a support structure of a foot articulation that is able to be proactive in the prevention of unnatural movements that causes injuries, and which is able to be a support structure conforms to sports activities.

It is another feature of the present invention to provide a support structure of a foot articulation, structurally easy and cheap to make.

It is also a feature of the present invention to provide a shoe that comprises said support structure of a foot articulation.

These and other objects are achieved by a support structure of a foot articulation comprising:

a body in which said foot articulation is adapted to be arranged;

a lung body, associated with said body, which defines a deformable chamber, said lung body comprising:

a first portion which extends in a substantially longitudinal direction along said foot in a laterally position with respect to a foot sole, said first portion extends substantially starting from the heel up to the metatarsus of sad foot; and

a second portion which extends in a substantially orthogonal direction with respect to said first portion, said second portion being arranged between said body and an ankle of said foot articulation, said second portion being fluidically connected to said first portion;

such that:

when a foot support during the gait is made with a direction substantially orthogonal to a ground plane, said lung body remains in an inactive configuration;

when a foot support during the gait is made with a direction inclined at an angle with respect to said ground plane, said lung body passes in an active configuration in which a first force on said first portion is generates, said first force being defined by a first vector substantially orthogonal with respect to said ground plane, said first force causing—on feedback—a subsequent expansion of said second portion that generates a second force, said second force being defined by a second vector substantially parallel to said ground plane, such that a support for said ankle is provided.

In particular, the first and the second portion of the lung body are adapted to change in response to the actions of the foot, which is in contact with the ground plane. Therefore, when occurs an inclination or distortion of the foot on the outer or inner edge, the first portion is subject to compression; the second portion—fluidically connected to this—arranged between the ankle and the outer and/or inner edge of the shoe, it is able to expand generating a second force, orthogonal with respect to the ankle that protects, support and contain the ankle movements. In this way a substantial stiffening of the articulation of the foot that prevent serious distortions is provided.

This way, the lung body presents a proactive support adapted to protect and support the foot and the ankle during accidental movements that can cause actions potentially dangerous and injuries.

The support offered by the lung body is not permanent but is in the active configuration when occurs that the foot doesn't place in a substantially orthogonal position with respect to the plane ground, but rather at an angle with respect to the ground plane; the lung body go back in the inactive configuration when the foot doesn't place in the inclined position.

Preferably said body is defined by a shoe, a sock support, an ankle support, a sole support or the like.

Advantageously, said lung body contains in said deformable chamber a fluid with predetermined density. For example, a fluid selected from the group consisting of: a liquid, a gas, a gel or a combination thereof.

Preferably said lung body is conformed is conformed has a reverse-T shaped wherein the a base portion of the “T” is arranged in correspondence of the side edge of the foot, and wherein a stem portion of the “T” is arranged in correspondence of the ankle of the foot.

In particular, the stem of the T is arranged in a decentered position substantially at the end of the first portion, which in use is at the heel of the foot.

In a preferred exemplary embodiment, a first and a second lung body located opposite to each other whit respect to the foot articulation are provided.

Advantageously, a connection duct fluidically connects said first and second lung body to each other. In this way, the first lung body mainly acts when the foot places into the plane ground in an inclined position towards an outer edge of the foot, whereas the second lung body mainly acts when the foot place into the plane ground in an inclined position towards an inner edge.

In this way, when happens a foot support at an angle position with respect to the ground plane, independently from such as the two lung bodies interested, both of said first and second lung body pass in the active configuration.

In particular, a first force according to a first vector is produced, generated by the compression of one of the first portions in contact with the ground by the edge where the foot is unbalanced. The compression causes—in feedback—a subsequent contemporaneous expansion of the second portions of both of said first and second lung body that generate second respective forces which has defined by respective second vectors opposite to each other. According to the function, when occurs the unbalancing of the foot one of said first and/or second lung body is activated, which is that on which a foot pressure at an angle with respect to the ground is generated; consequently, also the second lung body by the dynamic fluid connection existing between the two lung bodies is activated, such that said respective second portions of these expand.

In particular, both of the second portions of the lung bodies is able to expand towards directions opposite to each other in order to embrace the ankle and stabilizing it.

According to another aspect of the invention is provided a shoe that comprises a body in which a foot articulation is adapted to be arranged and a support structure of said foot articulation, wherein said support structure comprises:

a lung body, associated with said body, which defines a deformable chamber, said lung body comprising:

a first portion which extends in a substantially longitudinal direction along said foot in a laterally position with respect to a foot sole, said first portion extends substantially starting from the heel up to the metatarsus of sad foot; and

a second portion which extends in a substantially orthogonal direction with respect to said first portion, said second portion being arranged between said body and an ankle of said foot articulation, said second portion being fluidically connected to said first portion;

such that:

when a foot support during the gait is made with a direction substantially orthogonal to a ground plane, said lung body is in an inactive configuration;

when a foot support during the gait is made with a direction inclined at an angle with respect to said ground plane, said lung body passes in an active configuration in which a first force on said first portion is generates, said first force being defined by a first vector substantially orthogonal with respect to said ground plane, said first force causing—on feedback—a subsequent expansion of said second portion that generates a second force, said second force being defined by a second vector substantially parallel to said ground plane, such that a support for said ankle is provided.

Advantageously, said first portion is arranged at the edge of the tread shoe.

Preferably said first portion protrudes with respect to said edge of the tread shoe defining a pillow.

Advantageously, said second portion is arranged at an inner wall of said body arranged between the upper and the lining inner of said shoe.

In particular, the first portion in a cross sectional view has comma-shaped according to the profile of the perimetral shoe edge.

According to another aspect of the invention is supplied a sock support or an ankle support that comprises said support structure of the foot articulation.

In particular, the first portion of the lung body is arranged along the edge of the tread of the sock or ankle support and the second portion is arranged on the side face of the sock or ankle support and, preferably incorporate into the tissue of it.

According to another aspect of the invention an insole that comprises said support structure of the foot articulation is provided.

### DESCRIPTION OF FIGURES OF THE INVENTION

Further characteristic and advantages of the invention are better shown by the examination of the following detailed description of more forms of preferred embodiment, but not exclusive, shown for example and not limitative, with the support of the attached drawings, wherein:

FIG. 1 shows a diagrammatical cross sectional view of a support structure of a foot articulation, according to the present invention;

FIGS. 2 and 3 show a perspective view of a shoe equipped with the support structure of FIG. 1, having a single lung body;

FIGS. 4 and 5 show respectively an elevational side view and a rear view of the shoe of FIGS. 2 and 3;

FIGS. 6 and 7 show the support structure of FIG. 1 applied to a sock support/ankle support;

FIG. 8 shows an elevational front view of a further exemplary embodiment of the shoe of FIGS. 2 and 3 having a first and a second lung body;

FIG. 9 shows a rear view of the shoe of FIG. 8 where advantageously, a dynamic connection fluid between the two lung bodies side that is arranged on the rear outer edge of the heel is provided.

### DETAILED DESCRIPTION OF THE INVENTION

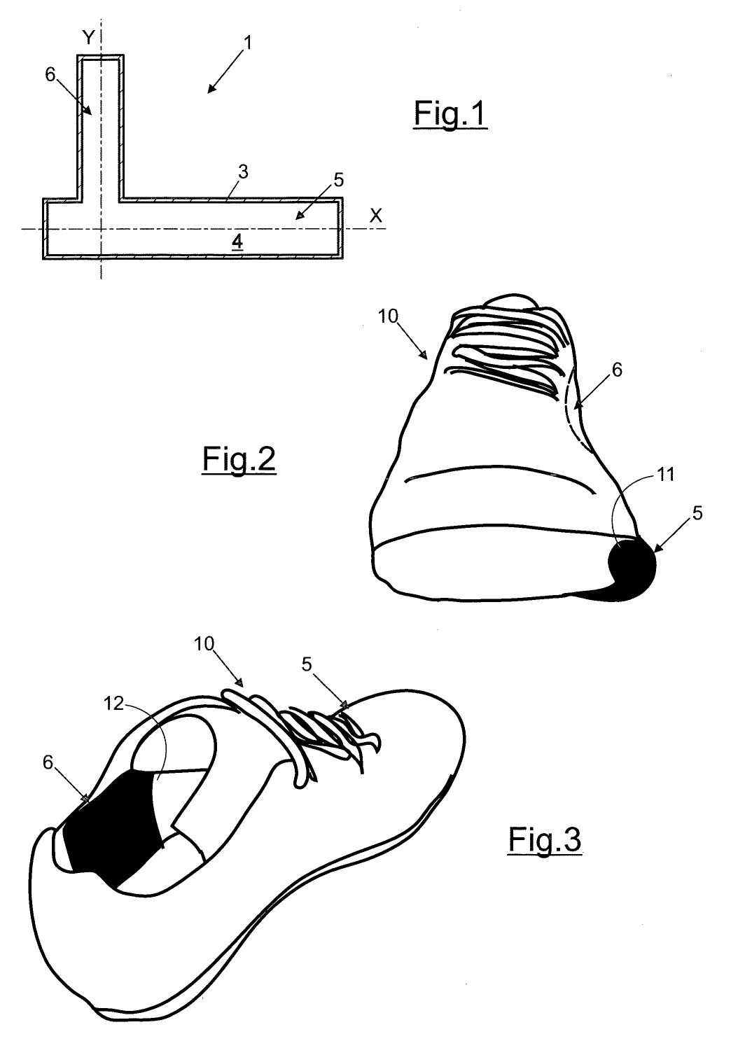

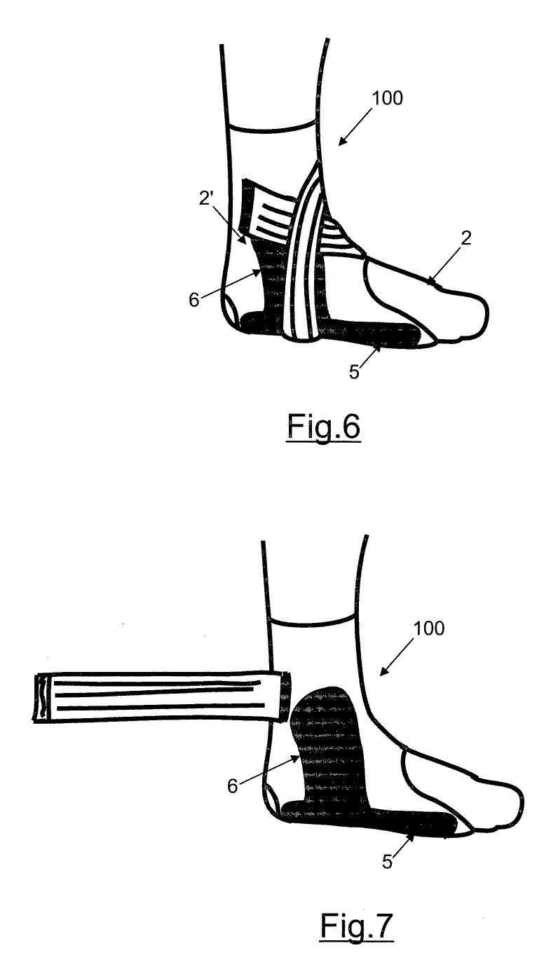

With reference to FIG. 1, is shown in a shape diagrammatical view a support structure 1 for a foot articulation 2 (FIG. 6). The support structure 1 comprises a body 1′ in which foot articulating 2 in adapted to be arranged, said support structure 1 also a lung body 3 that defines a deformable closed chamber 4. The lung body 4 is defined by a first portion 5, 5′ that extends in a direction substantially longitudinal along the axis X of the foot 2 from the heel to the metatarsus, and by a second portion 6, 6′ that extends in a direction substantially orthogonal along the axis Y with respect to the first portion 5 and is confined—in use—between the body 1′ and the ankle 2′(FIG. 6) of the foot 2.

This way, when a foot support during the gait maintain a normal direction substantially orthogonal with respect to the ground plane P the lung body remains in an inactive configuration A. When occurs a foot support during the gait inclined at an angle α with respect to the ground plane P, as shown in FIG. 5, the lung body 3 passes through an active configuration B. It is generates a first force according to a first vector T1, produced by the compression of the first portion 5 of the deformable chamber of the lung body 3, which in the contact with the ground, causes—in feedback—a subsequent expansion of the second portion 6. The second portion 6 being confined between the shoe and the ankle, generates a second force defined by a second vector T2 orthogonal with respect to the ankle 2′(FIG. 5). Therefore, the second portion 6 while expanding creates a side support for ankle 2′ in order to limit potential distortive effects of the incorrect foot support in a position not coplanar with respect to the ground plane P.

In other words, the first 5 and the second portion 6 of the lung body 3 is able to create a stiffening that embraces the foot 2 and the ankle 2′ in response to the foot actions 2 in contact with the ground plane P. In this way, the lung body 3 presents a proactive protection support which is adapted to support the foot 2 and the ankle 2′ during involuntary movements that can be determining potentially dangerous actions causes of injuries. In particular, the lung body 3 contains in the deformable chamber 4 a fluid. For example, a fluid selected from the group consisting of: a liquid, a gas, a gel or a combination thereof.

Structurally, as shown in FIG. 1 and in FIGS. 6 and 7, the lung body 3 is conformed has a reverse-T shaped with the a base portion of the “T” is arranged at the side edge of the foot 2, and whereas the stem portion of the “T” is arranged in correspondence to the ankle 2′. More in particular, the stem portion of the T, i.e. the second portion 6, is arranged in a decentered position with respect to the first portion 5, substantially at the end of the first portion 5, which in use is located at the heel of the foot 2.

In the exemplary embodiment, as shown in figures from 2 to 5, the support structure 1 is applied to a body 1′ of a shoe 10. The first portion 5 is arranged at a tread edge 11 of the shoe 10, whereas the second portion 6 is arranged at an inner wall 12 of the shoe 10 arranged between the upper and the lining internal.

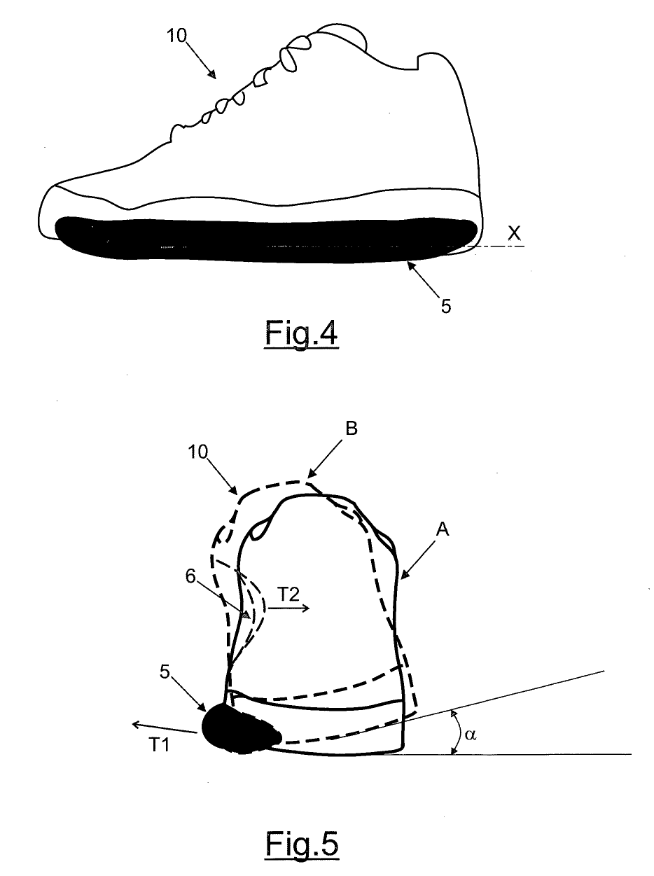

Preferably, as better shown in FIGS. 4 and 5, the first portion 5 protrudes with respect to the tread edge 11 defining a kind of side pillow.

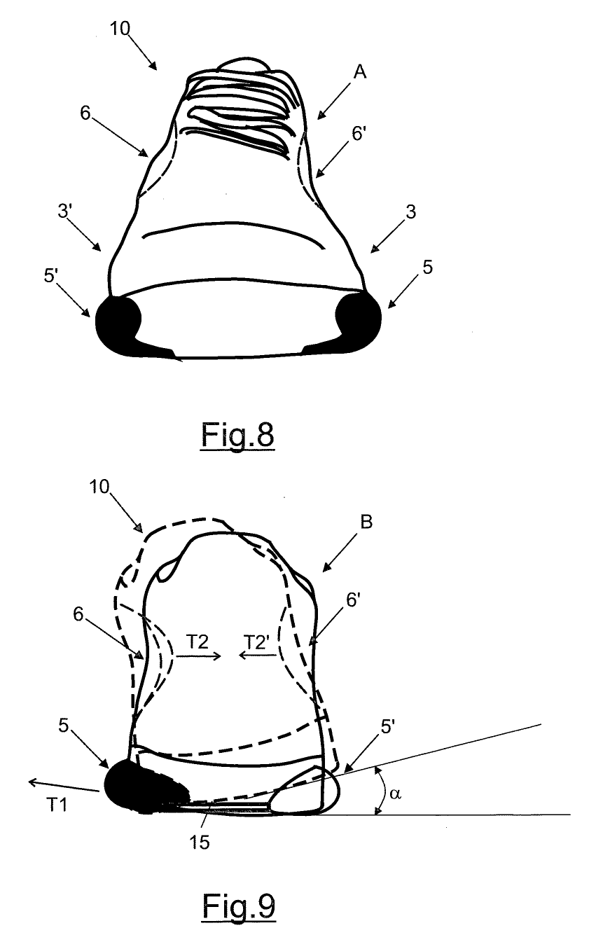

In particular, the first portion 5 in a cross sectional view has a comma-shaped profile. The first portion 5 comprises in particular, as better shown in FIG. 2, a first cross section 15 restricted which extends on either side of the tread edge 11, and a second cross section 16 neighboring to the first cross section 15 has a roundish-shaped profile. In an exemplary embodiment, as shown in FIGS. 8 and 9, a first 3 and a second lung body 3′ located opposite to each other whit respect to the foot articulation 2 are provided. The first lung body 3 acts mainly on the foot support, with respect to the ground P, which is inclined at an angle α towards the outer edge of the foot 2, whereas the second lung body 3′ acts mainly on the foot support, with respect to the ground plane P, which is inclined at an angle α towards the inner edge of the foot.

The first 3 and second lung body 3′, as better shown in FIG. 9, are fluidically connected to each other, by a connection duct 15 that extends starting from the respective first portions 5 and 5′. In particular, always as shown in FIG. 9, when occurs an unbalanced foot support indifferently towards the outer edge or inner edge, both the lung body 3 and 3′ pass in the active configuration B. In detail, the second portions 6 and 6′ are able to expand in opposite directions defined by vectors T2 and T2′ opposite to each other. Such effect allows to embrace the ankle and to limit the movements, reducing then the injuries risks.

In the FIGS. 6 and 7 an exemplary embodiment of the support structure applied to a sock support or ankle support 100 is shown. Also in this exemplary embodiment, the first portion 5 of the lung body 3 is arranged along the side edge of the sock support or ankle support and the second portion 6 is arranged on the side face of the sock support or ankle support 100 and, preferably it is incorporate into the tissue. Even in another exemplary embodiment, not shown, the support structure 1 is applied to an insole can be arranged in the shoe 10.

The description of which above of one or more exemplary embodiments particular is capable of show the invention from a viewpoint conceptual so that other, using the prior art, can be changing and/or adapting in various applications an exemplary embodiment without further researches and without moving away from each other by the inventive concept, and, then is intended that such adaptations and changes will be high as equivalent of an exemplary specific embodiment. The means and the material to provide the various functions described can be changes nature without for this come out from the field of invention. Is intended that the expresses or the terminology used have object purely described and for this not limitative.

1. A support structure (1) of a foot articulation (2) comprising:
a body (1′) in which said foot articulation (2) is adapted to be arranged;
a lung body (3, 3′), associated with said body (1′), which defines a deformable chamber (4),
wherein said lung body (3, 3′) comprises:
a first portion (5, 5′) that extends in a substantially longitudinal direction (X) along said foot (2) in a laterally position, wherein said first portion (5, 5′) extends substantially starting from a heel up to a metatarsus of said foot (2); and
a second portion (6, 6′) that extends in a substantially orthogonal direction (Y) with respect to said first portion (5, 5′), said second portion (6, 6′) being arranged between said body (1′) and an ankle (2′) of said foot articulation (2);
such that:
when a foot support during a gait is made with a direction substantially orthogonal to a ground plane (P), said lung body (3, 3′) is in an inactive configuration (A);
when a foot support during the gait is made with a direction inclined at an angle (α) with respect to said ground plane (P), said lung body (3, 3′) passes in an active configuration (B) in which a first force on said first portion (5, 5′) is generated, said first force being defined by a first vector (T1) substantially orthogonal with respect to said ground plane (P), said first force causing—on feedback—a subsequent expansion of said second portion (6, 6′) that generates a second force, said second force being defined by a second vector (T2) substantially parallel to said ground plane (P), such that a support for said ankle is provided.

2. The support structure (1) according to claim 1, wherein said lung body (3, 3′) contains in said deformable chamber (4) a fluid with predetermined density.

3. The support structure (1) according to claim 2, wherein said fluid is selected from the group consisting of: a liquid, a gas, a gel, a natural or synthetic rubber, a latex and combinations thereof.

4. The support structure (1) according to claim 1 wherein said lung body (3, 3′) has an inverted T-shape wherein a base portion of said “T” is arranged at the side edge of the foot (2) and wherein the stem portion of said “T” is arranged at the ankle (2′) of the foot (2), in particular said second portion (6, 6′) is arranged in a decentered position substantially at the end of said first portion (5, 5′), which in use is arranged in correspondence of the heel of said foot (2).

5. The support structure (1) according to claim 1, wherein a first (3) and a second lung body (3′) arranged opposite to each other with respect to said foot articulation (2) are provided.

6. The support structure (1) according to claim 5, wherein said first (3) and second lung (3′) bodies are fluidically independent from each other.

7. Support structure (1) according to claim 5, wherein said first (3) and second lung body (3′) are fluidically connected to each other by a connection duct (15), in such a way that:
when occurs a foot support during the gait with a direction inclined at an angle (α) with respect to the ground plane (P), the first (3) or second lung body (3′) on which a foot pressure is generated, passes in said active configuration causing, by the fluid dynamic connection made by said connection duct (15), activation of said second (3′) or first (3) lung body located on the other edge of the foot (2), such that both said first and second lung bodies (3, 3′) switch in said active configuration (B);
and such that a first force according to a first vector (T1) is generated, produced by compression of said first portion (5) or (5′) of the deformable chamber of the lung body (3) or (3′), which causes—in feedback—by the contact with the ground (P), a subsequent contemporaneous expansion of said second portions (6, 6′) that generate respective second forces defined by second vectors (T2, T2′) which are opposite to each other.

8. A shoe (10) comprising a body (1′) in which a foot articulation (2) can be arranged, and a support structure (1) of the foot articulation (2), wherein said support structure (1) of the foot articulation comprises:
a lung body (3, 3′), associated with said body (1′), which defines a deformable chamber (4),
wherein said lung body (3, 3′) comprises:
a first portion (5, 5′) that extends in a substantially longitudinal direction (X) along said foot (2) in a laterally position, wherein said first portion (5, 5′) extends substantially starting from a heel up to a metatarsus of said foot (2); and
a second portion (6, 6′) that extends in a direction substantially orthogonal direction (Y) with respect to said first portion (5, 5′), said second portion (6, 6′) being arranged between said body (1′) and an ankle (2′) of said foot articulation (2);
such that:
when a foot support during a gait is made with a direction substantially orthogonal to a ground plane (P), said lung body (3, 3′) is in an inactive configuration (A);
when a foot support during the gait is made with a direction inclined at an angle (α) with respect to said ground plane (P), said lung body (3, 3′) passes in an active configuration (B) in which a first force on said first portion (5, 5′) is generated, said first force being defined by a first vector (T1) substantially orthogonal with respect to said ground plane (P), said first force causing—on feedback—a subsequent expansion of said second portion (6, 6′) that generates a second force, said second force being defined by a second vector (T2) substantially parallel to said ground plane (P), such that a support for said ankle is provided.

9. The shoe (10) according to claim 8, wherein said first portion (5, 5′) is arranged at a tread edge (11) of said shoe (10) and said second portion (6, 6′) is arranged at an inner wall (12) of said body (1′) which is arranged between the upper and the lining inner of said shoe, said first portion (5, 5′) protrudes with respect to said tread edge (11) in order to define a side pillow.

10. The shoe (10) according to claim 8, wherein first (3) and a second (3′) lung bodies are provided located opposite to each other in foot articulation (2).

11. The shoe (10) according to claim 10, wherein said first (3) and second lung (3′) bodies are fluidically independent from each other.

12. The shoe (10) according to claim 10, wherein said first (3) and second (3′) lung bodies are fluidically connected to each other by a connection duct (15), in such a way that:
when a foot support occurs during the gait with a direction inclined at an angle (α) with respect to the ground plane (P), the first (3) or second (3′) lung body on which a foot pressure is generated, passes in said active configuration causing, by the fluid dynamic connection made by said connection duct (15), activation of said second (3′) or first (3) lung body located on the other edge of the foot (2), such that both said first and second lung body (3, 3′) switch in said active configuration (B);
and such that a first force according to a first vector (T1) is generates, produced by compression of said first portion (5) or (5′) of the deformable chamber of the lung body (3) or (3′), which causes—in feedback—by contact with the ground (P), a subsequent contemporaneous expansion of said second portions (6, 6′) that generate respective second forces defined by second vectors (T2, T2′) which are opposite to each other.

13. A sock support or ankle support (100) comprising a support structure (1) of the foot articulation (2) according to claim 1.

14. An insole that comprising a support structure (1) of the foot articulation (2) according to claim 1.
